(19) United States
(12) Patent Application Publication
(10) Pub. No.: US 2013/0079714 A1
(43) Pub. Date: Mar. 28, 2013

(54) SINGLE USE SYRINGE

(71) Applicant: Ming-Yuan WU, Taipei City (TW)

(72) Inventor: Ming-Yuan WU, Taipei City (TW)

(21) Appl. No.: 13/243,093

(22) Filed: Sep. 23, 2011

(51) Int. Cl. A61M 5/50

(52) U.S. Cl. 604/110

(57) ABSTRACT

A single use syringe comprising a plunger arm, plunger tip, syringe body, and tip retaining sleeve is disclosed. The tip retaining sleeve surrounds tip retaining arms of the plunger arm allowing the tip retaining arms to grasp the plunger tip. When the plunger arm is pulled backwards, sleeve ribs of the tip retaining sleeve protrude into a sleeve lock of the syringe body and the tip retaining sleeve is prevented from moving in a forward direction. As the plunger arm is pushed forward, the tip retaining arms are no longer surrounded by the tip retaining sleeve and the tip retaining arms move outwards and release the plunger tip. After the plunger tip has been released from the plunger arm, the plunger tip and the plunger arm cannot be reconnected and the single use syringe cannot be reused.

### BACKGROUND OF THE INVENTION

1. Field of the Invention

The present invention relates to medical syringes. More specifically, the present invention discloses a safe and effective syringe that automatically ensures the syringe cannot be reused.

2. Description of the Prior Art

Syringes are commonly used medical implements for injecting medicine into a patient. A medical professional draws medicine into the syringe and then injects the medicine into a patient through a needle in the syringe.

While useful, the conventional syringe has various disadvantages that permit potentially dangerous use of the syringe. Misuse of the traditional syringe can be fatal or detrimental to a patient's health.

One major disadvantage of the conventional syringe is that the syringe can easily be used again. The conventional syringe or components of the syringe can easily be used again since the conventional syringe provides no inherent means to prevent reuse.

After a patient is injected with a medicine, residue such as blood, medicine, virus, bacteria, or other contaminants remain on the needle or inside the syringe. If another patient is injected using the used syringe, the second patient can be contaminated with residue from the first patient.

If the first patient is infected with a virus, the second patient is susceptible to contracting the virus from the previously used syringe. The second patient is also susceptible if the second patient is allergic to the medicine used with the first patient.

Additionally, it can be difficult to determine whether or not the syringe has been previously used.

Therefore, there is need for an improved medical syringe that effectively delivers medicine to a patient while automatically preventing reuse of the syringe.

### SUMMARY OF THE INVENTION

To achieve these and other advantages and in order to overcome the disadvantages of the conventional method in accordance with the purpose of the invention as embodied and broadly described herein, the present invention provides a syringe that automatically disconnects components of the syringe to prevent reuse.

The single use syringe of the present invention comprises a plunger arm, plunger tip, syringe body, and a tip retaining sleeve. The plunger arm comprises an arm post that is inserted into a mating hollow interior section of a tip base of the plunger tip. The tip retaining sleeve surrounds tip retaining arms of the plunger tip allowing the tip retaining arms to grasp the plunger tip.

When the plunger arm is pulled backwards, sleeve rings of the tip retaining sleeve press against the syringe body and the tip retaining sleeve is prevented from moving in a forward direction.

As the plunger arm is pushed forward, the tip retaining arms are no longer surrounded by the tip retaining sleeve and the tip retaining arms move outwards and release their grasp on the plunger arm. After the plunger tip has been released, the arm post of the plunger arm can continue to push the plunger tip forward until the end of the syringe body has been reached. However, after the plunger tip has been released, the plunger arm cannot grasp or pull the plunger tip backwards.

After the plunger tip has been released from the plunger arm, the plunger tip and the plunger arm cannot be reconnected and the single use syringe cannot be reused.

The plunger arm further comprises a used indicator. The used indicator comprises, for example, lettering or wording such as “USED” and/or a bright color. After the syringe has been used the used indicator allows easy confirmation that this syringe has been previously used. The used indicator is hidden and not visible until the syringe has been used.

Not only is the single use syringe of the present invention useful in preventing reuse of the syringe by medical professionals but also prevents addicts or intravenous drug users from sharing needles.

The single use syringe of the present invention dramatically decreases the need for needle exchange programs to ensure that intravenous drug users do not share needles. Even if the user does not return the used syringe to an exchange center, the single use syringe cannot be used by another addict.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings:

FIG. 1A is a drawing illustrating a cross-sectional view of a plunger arm of a single use syringe according to an embodiment of the present invention;

FIG. 1B is a drawing illustrating a side view of a plunger arm of a single use syringe according to an embodiment of the present invention;

FIG. 1C is a drawing illustrating an end view of a plunger arm of a single use syringe according to an embodiment of the present invention;

FIG. 1D is a drawing illustrating a cross-sectional view of a knob of a plunger arm of a single use syringe according to an embodiment of the present invention;

FIG. 1E is a drawing illustrating a side view of a knob of a plunger arm of a single use syringe according to an embodiment of the present invention;

FIG. 1F is a drawing illustrating an end view of a knob of a plunger arm of a single use syringe according to an embodiment of the present invention;

FIG. 2A is a drawing illustrating a cross-sectional view of a tip retaining sleeve of a single use syringe according to an embodiment of the present invention;

FIG. 2B is a drawing illustrating a side view of a tip retaining sleeve of a single use syringe according to an embodiment of the present invention;

FIG. 2C is a drawing illustrating an end view of a tip retaining sleeve of a single use syringe according to an embodiment of the present invention;

FIG. 3A is a drawing illustrating a cross-sectional view of a plunger tip of a single use syringe according to an embodiment of the present invention;

FIG. 3B is a drawing illustrating a side view of a plunger tip of a single use syringe according to an embodiment of the present invention;

FIG. 3C is a drawing illustrating an end view of a plunger tip of a single use syringe according to an embodiment of the present invention;

FIG. 4A is a drawing illustrating a cross-sectional view of a syringe body of a single use syringe according to an embodiment of the present invention;

FIG. 4B is a drawing illustrating a side view of a syringe body of a single use syringe according to an embodiment of the present invention;

FIG. 4C is a drawing illustrating an end view of a syringe body of a single use syringe according to an embodiment of the present invention;

FIG. 4D is a drawing illustrating a cross-sectional view of a sleeve stopper of a syringe body of a single use syringe according to an embodiment of the present invention;

FIG. 4E is a drawing illustrating a side view of a sleeve stopper of a syringe body of a single use syringe according to an embodiment of the present invention;

FIG. 4F is a drawing illustrating an end view of a sleeve stopper of a syringe body of a single use syringe according to an embodiment of the present invention;

FIG. 5A is a drawing illustrating a cross-sectional view of a single use syringe according to an embodiment of the present invention;

FIG. 5B is a drawing illustrating a side view of a single use syringe according to an embodiment of the present invention; and

FIGS. 6A-6H are drawings illustrating movement and operation of a single use syringe with sleeve stopper and end positioned body grasp flange according to an embodiment of the present invention.

### DESCRIPTION OF THE PREFERRED EMBODIMENTS

Reference will now be made in detail to the preferred embodiments of the present invention, examples of which are illustrated in the accompanying drawings. Wherever possible, the same reference numbers are used in the drawings and the description to refer to the same or like parts.

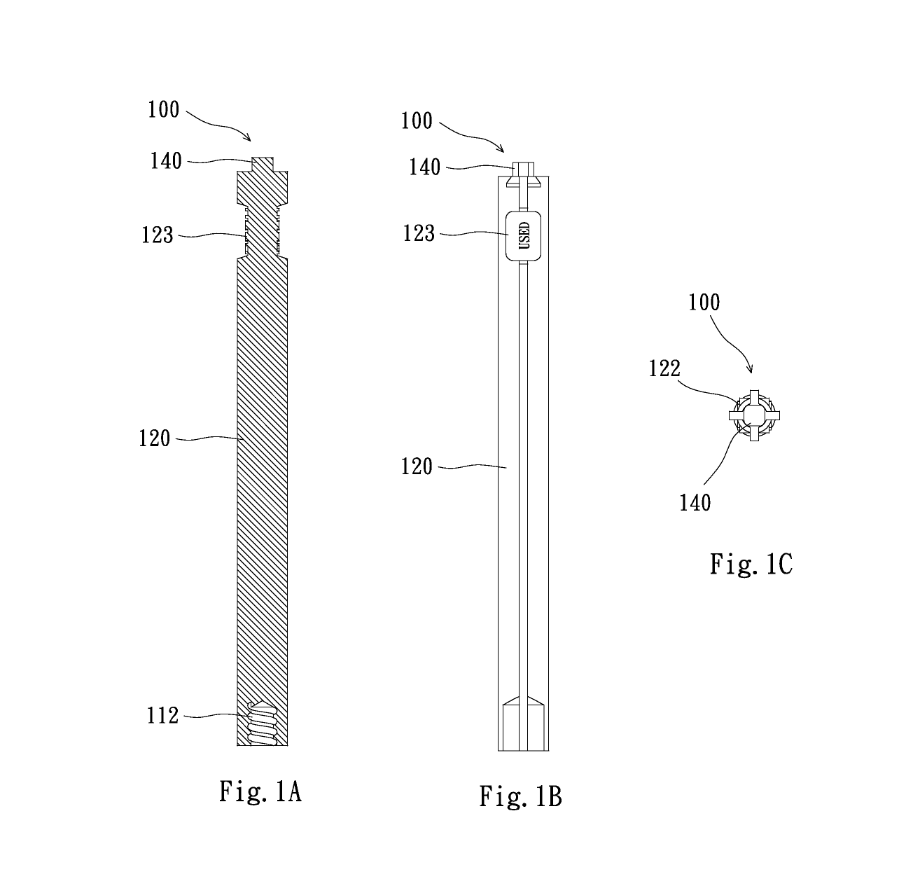

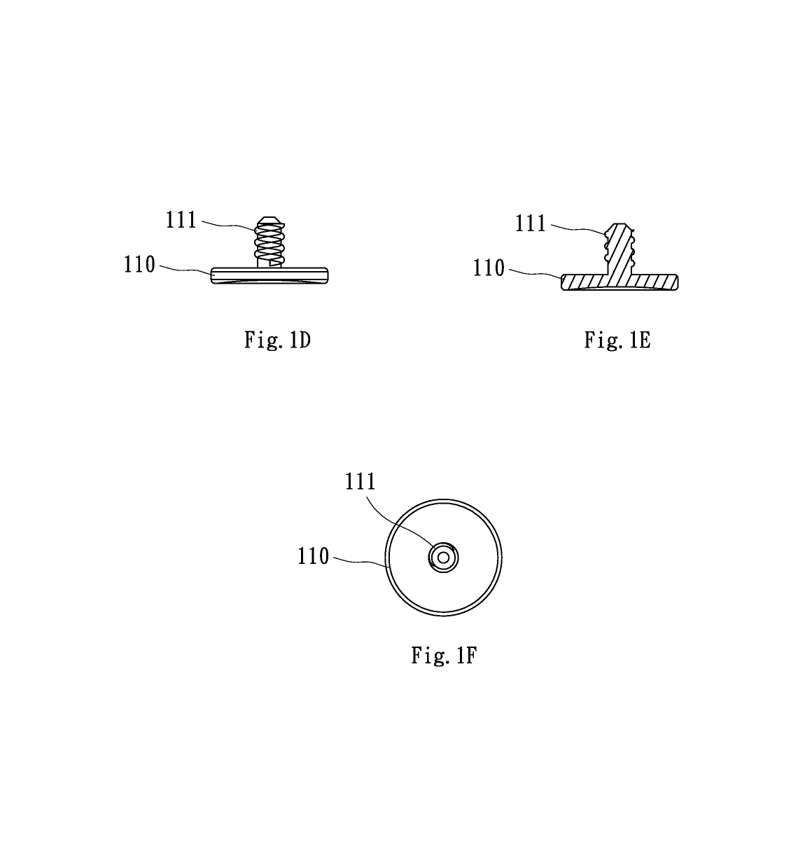

Refer to FIG. 1A, which is a drawing illustrating a cross-sectional view of a plunger arm of a single use syringe according to an embodiment of the present invention; to FIG. 1B, which is a drawing illustrating a side view of a plunger arm of a single use syringe according to an embodiment of the present invention; to FIG. 1C, which is a drawing illustrating an end view of a plunger arm of a single use syringe according to an embodiment of the present invention; to FIG. 1D, which is a drawing illustrating a cross-sectional view of a knob of a plunger arm of a single use syringe according to an embodiment of the present invention; to FIG. 1E, which is a drawing illustrating a side view of a knob of a plunger arm of a single use syringe according to an embodiment of the present invention; and to FIG. 1F, which is a drawing illustrating an end view of a knob of a plunger arm of a single use syringe according to an embodiment of the present invention.

The single use syringe of the present invention comprises a plunger assembly, a tip retaining sleeve, and a syringe body. The plunger assembly comprises a plunger arm and a plunger tip that removably attached together. The plunger assembly allows medicine to be drawn into the syringe body and the medicine to be injected into a patient.

As shown in FIGS. 1A-1F the plunger arm 100 comprises a plunger knob 110, a plunger stem 120, tip flange 122, and an arm post 140.

In an embodiment of the present invention the plunger knob 110 further comprises a threaded tip 111 that allows the plunger knob 110 to be screwed into stem threads 112 disposed in the end of the plunger stem 120.

In another embodiment of the present invention the plunger knob 110 is ultrasonically welded to the plunger stem 120.

The plunger knob 110 is positioned on one end of the plunger stem 120. The plunger knob 110 allows a medical professional or user of the syringe to pull back the plunger arm 100 of the plunger assembly in order to draw liquid into the syringe. The plunger knob 110 also allows a professional or user of the syringe to push on the plunger knob 110 in order to move the plunger assembly forward and expel liquid from the syringe.

Positioned on the other end of the plunger stem 120, the plunger arm 100 comprises at least one tip flange 122 and an arm post 140. The tip flange 122 and the arm post 140 cooperate with the plunger tip to position and hold the plunger tip of the plunger assembly.

The plunger stem further comprises a used indicator 123. The used indicator 323 comprises, for example, lettering or wording such as “USED” and/or a bright color. After the syringe has been used the used indicator 123 allows a user to easily identify that this syringe has been previously used. The used indicator 123 is hidden and not visible until the syringe has been used.

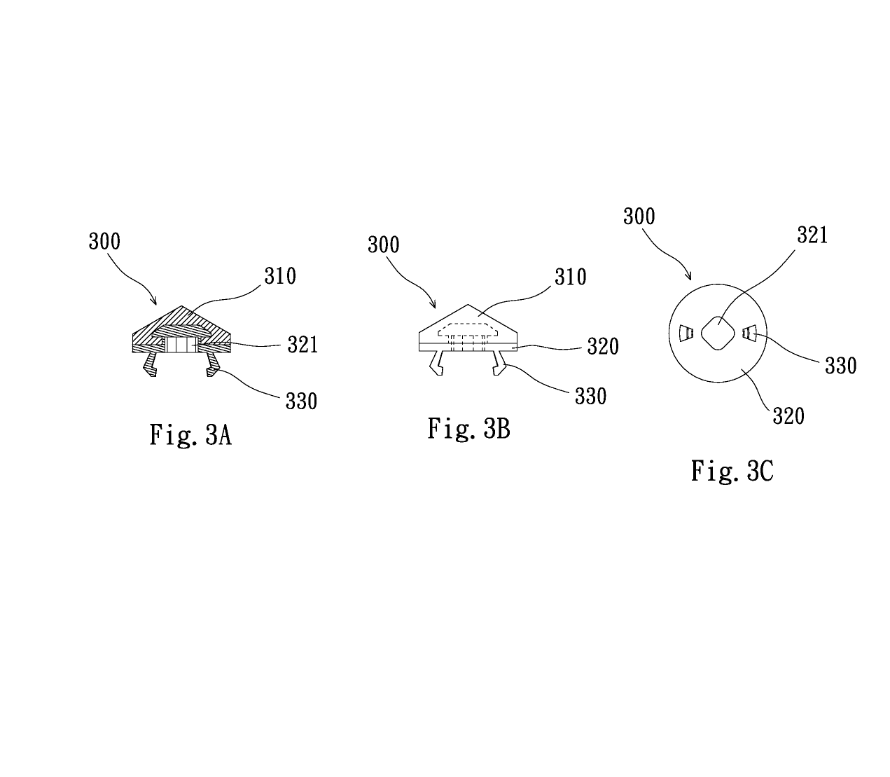

Refer to FIG. 3A, which is a drawing illustrating a cross-sectional view of a plunger tip of a single use syringe according to an embodiment of the present invention, to FIG. 3B, which is a drawing illustrating a side view of a plunger tip of a single use syringe according to an embodiment of the present invention and to FIG. 3C, which is a drawing illustrating an end view of a plunger tip of a single use syringe according to an embodiment of the present invention.

The plunger assembly of the single use syringe of the present invention further comprises a plunger tip 300 that connects with the plunger arm.

The plunger tip 300 comprises a tip head 310 and a tip base 320. The tip base 320 comprises a hollow interior area 321 that mates with the arm post (140 FIG. 1A). The tip base 320 further comprises tip retaining arms 330 to grasp the tip flange (122 FIG. 1C) and hold the plunger tip 300 to the plunger arm.

In an embodiment of the present invention the hollow interior area 321 and the arm post are formed in a mating non-round shape to prevent the plunger tip 300 from rotating separately from the plunger arm. For example, the arm post and hollow interior area 321 are square, triangle, or diamond shaped. As a result, when the arm post is inserted into the hollow interior area 321 the plunger tip 300 is only allowed to rotate together with the plunger arm.

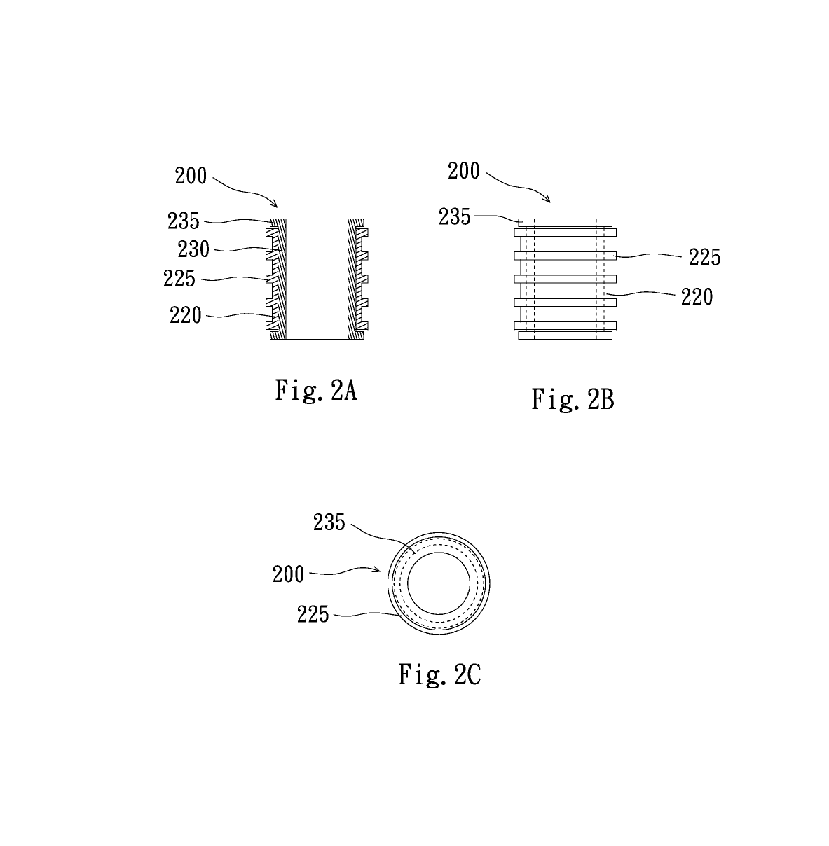

Refer to FIG. 2A, which is a drawing illustrating a cross-sectional view of a tip retaining sleeve of a single use syringe according to an embodiment of the present invention, to FIG. 2B, which is a drawing illustrating a side view of a tip retaining sleeve of a single use syringe according to an embodiment of the present invention, and to FIG. 2C, which is a drawing illustrating an end view of a tip retaining sleeve of a single use syringe according to an embodiment of the present invention.

The single use syringe of the present invention further comprises a tip retaining sleeve 200. The tip retaining sleeve 200 is an elongated tube that is positioned around the plunger arm. A plurality of sleeve ribs 225 are disposed on the tip retaining sleeve 200 and extend outside of the main body of the tip retaining sleeve 200.

In an embodiment of the present invention the inside surface of the tip retaining sleeve 200 is angled or has a chamfered end to allow the tip retaining arms to exit the tip retaining sleeve more easily and release the plunger tip sooner.

In an embodiment of the present invention the tip retaining sleeve 200 comprises an inner plastic sleeve 230 and an outer rubber sleeve 220. The plastic sleeve 230 is disposed inside the rubber sleeve 220. Tabs 235 on the ends of the plastic sleeve 230 hold the rubber sleeve 220 to the plastic sleeve 230 and prevent the rubber sleeve 220 from sliding off of the plastic sleeve 230.

The inner plastic sleeve 230 reduces friction between the tip retaining sleeve 200 and the plunger arm. The sleeve ribs 225 of the outer rubber sleeve 220 cooperate with the syringe body wall to prevent forward movement of the tip retaining sleeve 200.

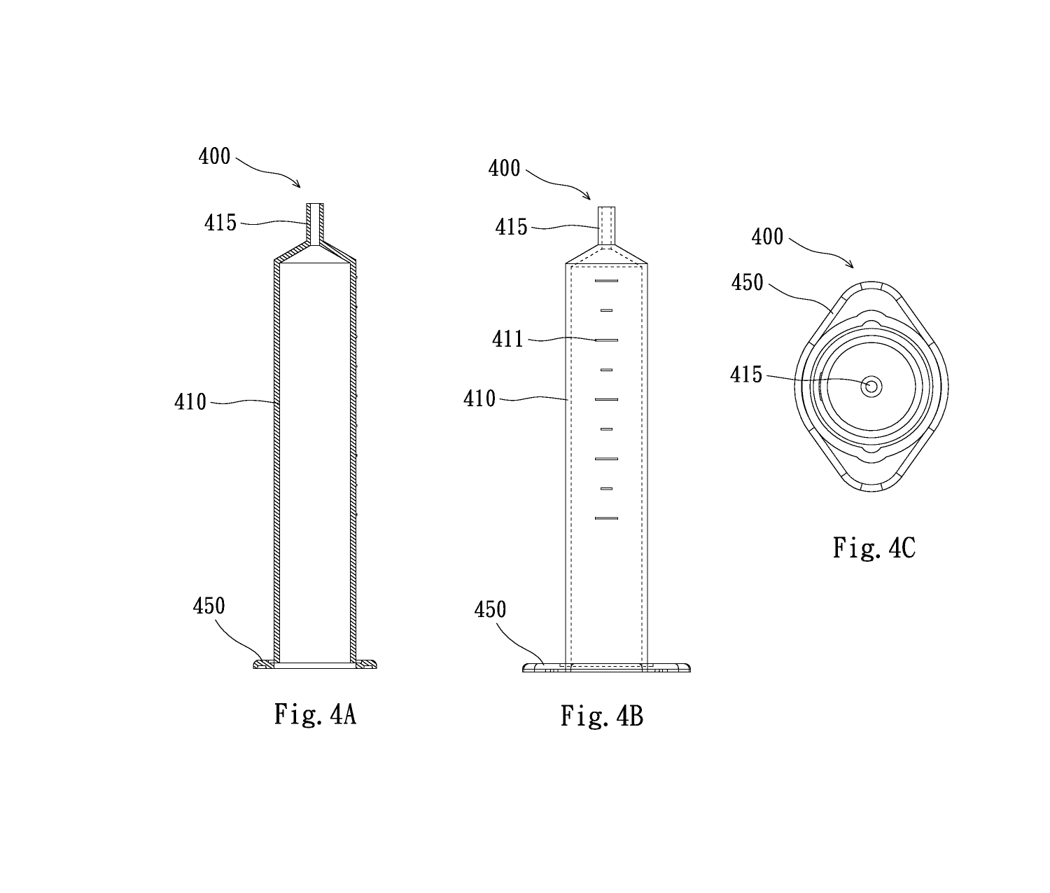

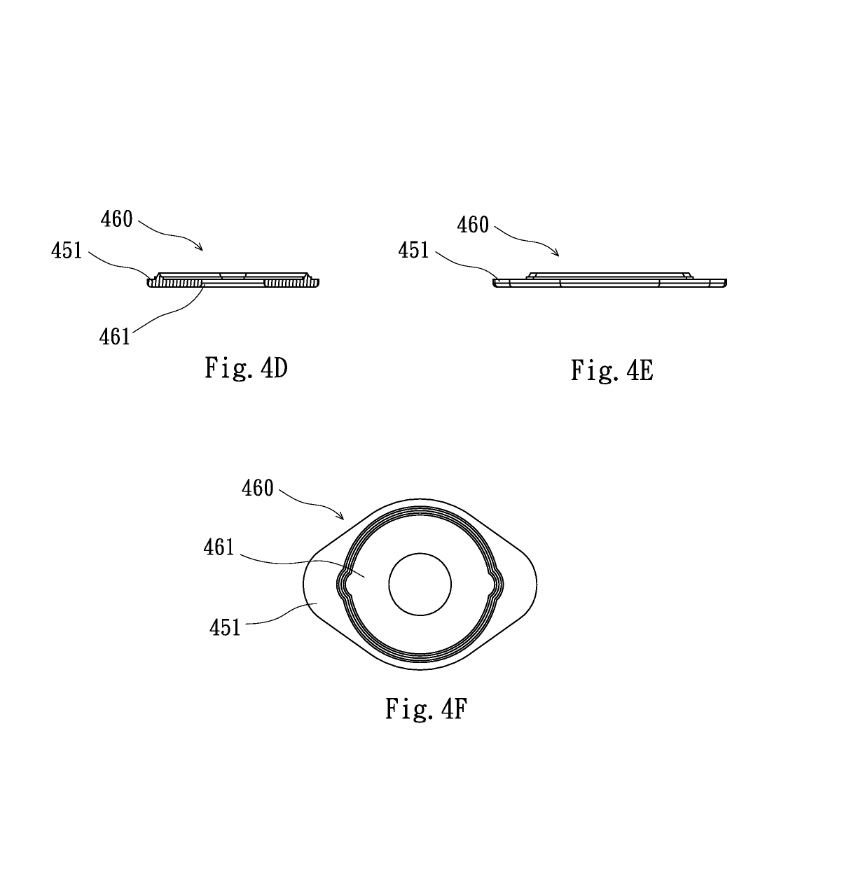

Refer to FIG. 4A, which is a drawing illustrating a cross-sectional view of a syringe body of a single use syringe according to an embodiment of the present invention and to FIG. 4B, which is a drawing illustrating a side view of a syringe body of a single use syringe according to an embodiment of the present invention and to FIG. 4C, which is a drawing illustrating an end view of a syringe body of a single use syringe according to an embodiment of the present invention and to FIG. 4D, which is a drawing illustrating a cross-sectional view of a sleeve stopper of a syringe body of a single use syringe according to an embodiment of the present invention and to FIG. 4E, which is a drawing illustrating a side view of a sleeve stopper of a syringe body of a single use syringe according to an embodiment of the present invention and to FIG. 4F, which is a drawing illustrating an end view of a sleeve stopper of a syringe body of a single use syringe according to an embodiment of the present invention.

The single use syringe of the present invention further comprises a syringe body 400. The syringe body 400 comprises a syringe body tip 415, a syringe body wall 410, a body grasp flange 450, and a sleeve stopper 460.

A needle for injecting a patient is molded into the syringe body tip 415.

The body grasp flange 450 extends outwardly from the syringe body 400 to assist a professional or user during operation of the syringe. To aid in pulling back the plunger assembly the syringe user places a finger or fingers on the top side of the body grasp flange 450 while pulling back on the plunger knob. When pressing on the plunger knob to move the plunger assembly forward the syringe user places a finger or fingers on the bottom side of the body grasp flange 450.

The sleeve stopper 460 comprises a second body grasp flange 451 that mates with the first body grasp flange 450 and a sleeve stopper wall 461 that prevents the tip retaining sleeve (200 FIG. 2A 2B) from exiting the syringe body 400.

The syringe body 400 further comprises volume markers 411 that allow indication of the volume of medicine or fluid contained in the syringe body 400.

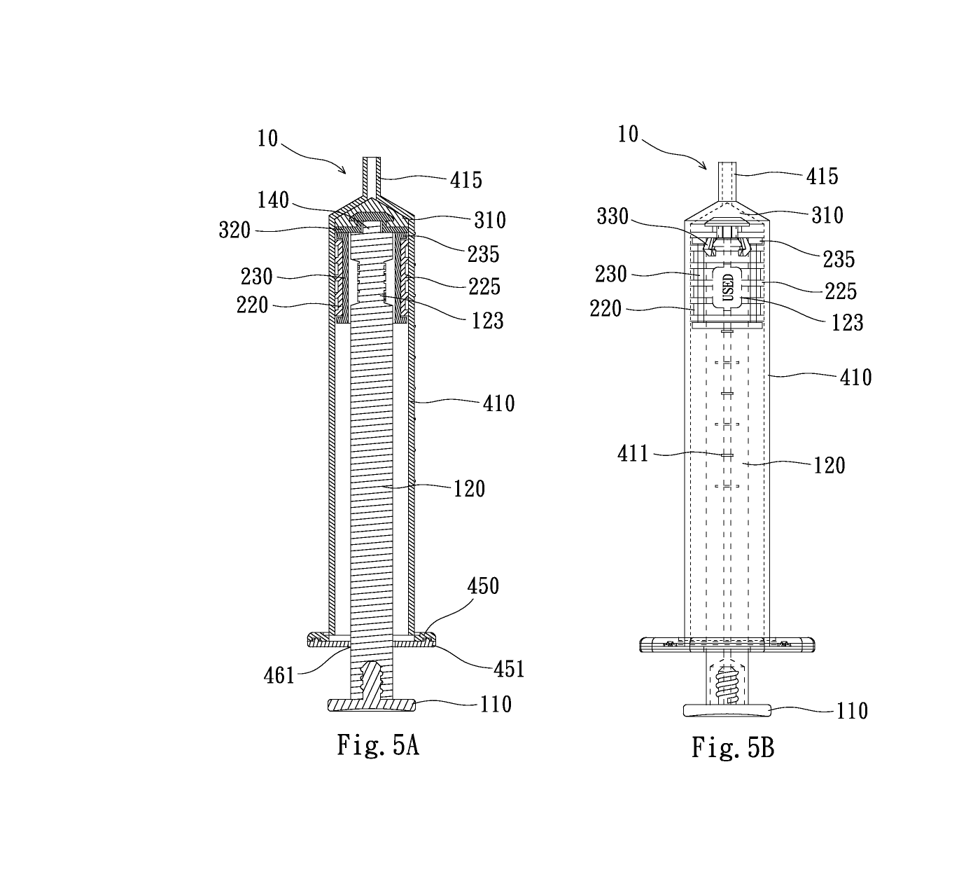

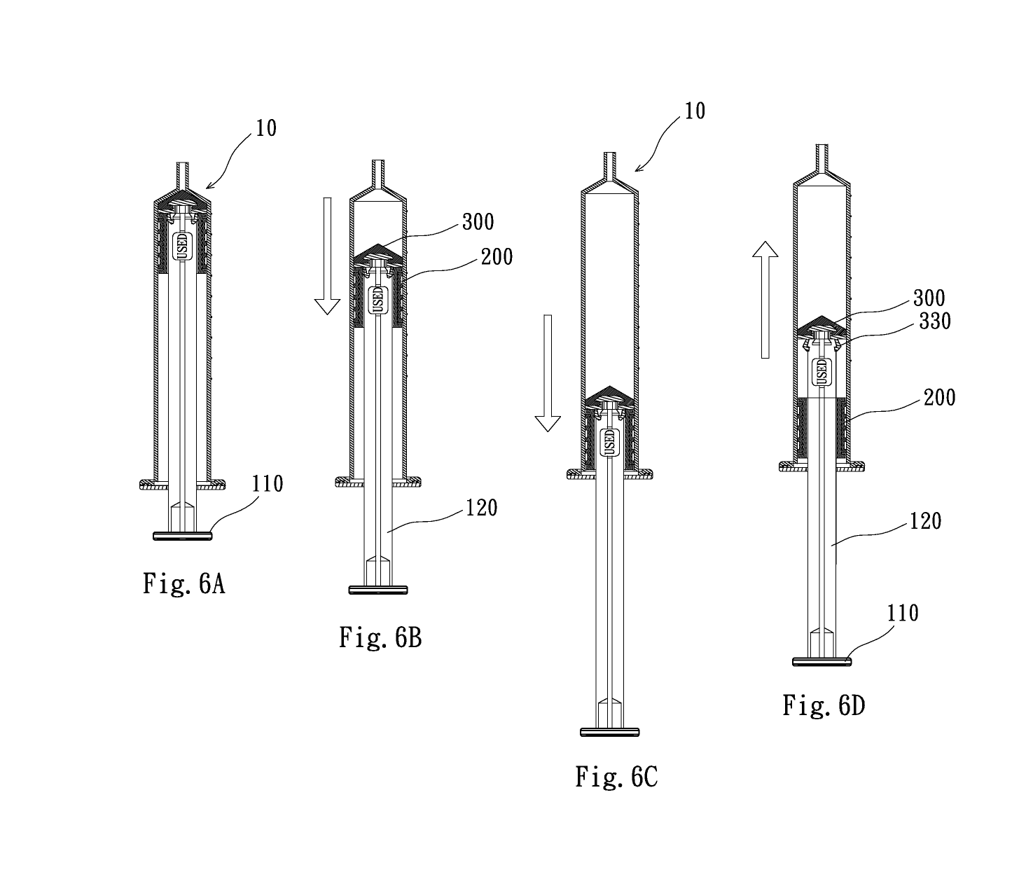

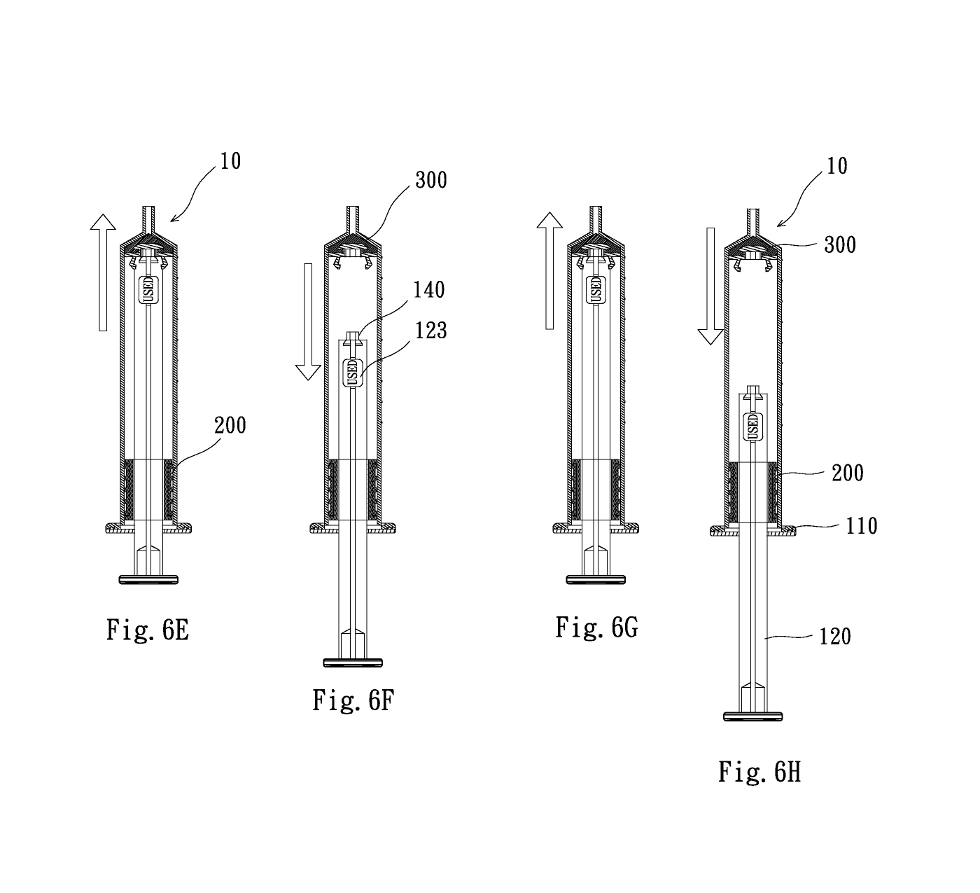

Refer to FIG. 5A, which is a drawing illustrating a cross-sectional view of a single use syringe according to an embodiment of the present invention and to FIG. 5B, which is a drawing illustrating a side view of a single use syringe according to an embodiment of the present invention and to FIGS. 6A-6H, which are drawings illustrating movement and operation of a single use syringe with sleeve stopper and end positioned body grasp flange according to an embodiment of the present invention. Also, continue to refer to FIGS. 1A-4F.

Prior to use, the elements of the single use syringe 10 of the present invention are positioned as shown in FIGS. 5A and 5C. The plunger tip 300 is positioned at the forward area of the interior of the syringe body 400. The arm post 140 of the plunger arm 100 is positioned inside the base opening 321 of the tip base 320 of the plunger tip 300. The tip retaining sleeve 200 surrounds the tip retaining arms 330 of the plunger tip 300 and holds the tip retaining arms 330 grasping the tip flange 122 to hold the plunger tip 300 and the plunger arm 100 together.

When assembled, the single use syringe 10 of the present invention comprises the plunger assembly (plunger arm 100 and plunger tip 300) and the tip retaining sleeve 200 disposed inside the syringe body 400.

As the plunger arm 100 is pulled back the tip retaining sleeve 200 holds the tip retaining arms 330 in to grasp the tip flange 122 so that the plunger tip 300 is pulled back along with the plunger arm 100. As the plunger tip 300 is pulled back the plunger head 310 pulls the tip retaining sleeve 200 back with the plunger tip 300.

As the tip retaining sleeve 200 moves backwards in the syringe body 400, the sleeve ribs 225 of the tip retaining sleeve 200 contact the syringe body wall 410 of the syringe body 400. At any time after backward movement, the friction between the sleeve ribs 225 and the interior surface of the syringe body wall 410 prevents the tip retaining sleeve 200 from moving in the forward direction.

As the plunger arm 100 continues to be pulled backward the tip retaining sleeve 200 continues to move backwards along with the plunger assembly (plunger arm 100 and plunger tip 300).

When the tip retaining sleeve 200 reaches the rear opening of the syringe body 400, the sleeve stopper 460 of the syringe body 400 blocks the backward movement of the tip retaining sleeve 200 and prevents the tip retaining sleeve 200 from exiting the syringe body 400. At this position the tip retaining sleeve 200 is prevented from moving backward by the sleeve stopper 460 and prevented from moving forward by the sleeve ribs 225 pressing against the interior wall of the syringe body 400.

When the plunger knob 110 is pushed the plunger arm 100 moves forward. Since the tip retaining sleeve 200 is prevented from moving forward, the tip retaining arms 330 move outwards as the tip retaining sleeve 200 no longer surrounds the tip retaining arms 330. After the tip retaining arms 330 have moved outwards and away from the plunger arm 100, the tip retaining arms 330 no longer grasp the tip flange 122 of the plunger arm 100. However, the plunger tip 300 can continue to be moved in a forward direction since it is now pushed by the arm post 140.

When the plunger assembly has been moved all the way forward, the forward end of the syringe body 400 stops forward movement of the plunger tip 300.

When the plunger arm 100 is now pulled backwards the plunger tip 300 is separated from the plunger arm 100 since the tip retaining arms 330 no longer grasp the tip flange 122 of the plunger arm 100.

After the plunger tip 300 has been released from the plunger arm 100, the plunger arm 100 cannot reconnect with the plunger tip 300 and the single use syringe 10 cannot be used again.

At any time after the tip retaining arms 330 are not surrounded by the tip retaining sleeve 200 and have moved outwards, the plunger tip 300 has been released from the plunger arm 100 and the plunger tip 300 can no longer be pulled backwards by the plunger arm 100.

After the plunger arm 100 has moved forward the used indicator 123 of the plunger arm 100 provides visual identification and confirmation that the syringe has been used.

Utilizing a plurality of sleeve ribs 225 allows different dosage amounts with a single sized syringe while preventing reuse of the syringe. For example, if the dose requires the plunger assembly to travel backwards 25% of the interior of the syringe body, the sleeve ribs 225 prevent the tip retaining sleeve 200 from moving forward and the plunger tip 300 is released from the plunger arm 100. If the dose requires the plunger assembly to travel backwards 50% of the interior of the syringe body, the sleeve ribs 225 prevent the tip retaining sleeve 200 from moving forward and the plunger tip 300 is released from the plunger arm 100.

It will be apparent to those skilled in the art that various modifications and variations can be made to the present invention without departing from the scope or spirit of the invention. In view of the foregoing, it is intended that the present invention cover modifications and variations of this invention provided they fall within the scope of the invention and its equivalent.

What is claimed is:

1. a single use syringe comprising:
a plunger arm;
a plunger tip;
a sleeve, the sleeve surrounding a portion of the plunger arm and a portion of the plunger tip; and
a syringe body; the plunger arm, the plunger tip, and the sleeve disposed inside the syringe body;
wherein, after the plunger arm has been partially retracted the plunger tip is released from the plunger arm when the plunger tip exits the sleeve on forward movement of the plunger arm.

2. The single use syringe of claim 1, the plunger tip comprising a plurality of tip retaining arms for grasping a retaining flange on the plunger arm.

3. The single use syringe of claim 1, the sleeve comprising a plurality of ribs disposed on an outer surface of the sleeve.

4. The single use syringe of claim 1, the sleeve comprising an inner plastic sleeve and an outer rubber sleeve.

5. The single use syringe of claim 1, the syringe body further comprising a sleeve stopper for preventing the sleeve from exiting the syringe body.

6. The single use syringe of claim 1, the plunger arm comprising a used indicator for indicating the single use syringe has been used.

7. The single use syringe of claim 1, the plunger arm further comprising a post for inserting the plunger arm into the plunger tip.

8. The single use syringe of claim 7, wherein the post and the plunger tip are keyed in a mating configuration to prevent the plunger arm and the plunger tip from rotating independently.

9. A syringe comprising:
a plunger tip, the plunger tip comprising at least one tip retaining arm;
a plunger arm, the plunger arm removably attached to the plunger tip by the at least one tip retaining arm;
a tip retaining sleeve surrounding the at least one tip retaining arm; and
a syringe body holding the plunger arm, the plunger tip, and the tip retaining sleeve.

10. The syringe of claim 9, the tip retaining sleeve comprising at least one sleeve rib for preventing forward movement of the tip retaining sleeve.

11. The syringe of claim 9, where the plunger tip remains inside the syringe body before and after use.

12. The syringe of claim 9, where the at least one tip retaining arm releases the plunger tip after exiting the tip retaining sleeve.

13. The syringe of claim 9, the syringe body further comprising:
a sleeve stopper for preventing the tip retaining sleeve from exiting the syringe body.

14. The syringe of claim 9, where the at least one tip retaining arm exits the tip retaining sleeve during forward movement of the plunger arm.

15. The single use syringe of claim 9, the plunger arm comprising a used indicator for indicating the single use syringe has been used.

16. A syringe comprising:
a plunger arm;
a plunger tip, the plunger tip comprising at least one tip retaining arm for grasping the plunger arm;
a tip retaining sleeve comprising at least one sleeve rib, the tip retaining sleeve surrounding the at least one tip retaining arm when the at least one tip retaining arm is grasping the plunger arm; and
a syringe body surrounding the plunger arm, the plunger tip, and the tip retaining sleeve, the syringe body comprising a sleeve stopper for preventing the tip retaining sleeve from exiting the syringe body.

17. The syringe of claim 16, where the plunger tip remains inside the syringe body before and after use.

18. The syringe of claim 16, where the at least one tip retaining arm releases the plunger tip after exiting the tip retaining sleeve.

19. The syringe of claim 16, the syringe body further comprising:
a sleeve stopper for preventing the tip retaining sleeve from exiting the syringe body.

20. The syringe of claim 16, where the at least one tip retaining arm exits the tip retaining sleeve during forward movement of the plunger arm.
